(19) United States
(12) Patent Application Publication
(10) Pub. No.: US 2021/0128292 A1
(43) Pub. Date: May 6, 2021

(54) A FENESTRATED ENDOPROSTHESIS FOR THE CORRECTION OF AORTIC ANEURYSMS

(71) Applicant: UNIVERSITA' DEGLI STUDI DI PADOVA, Padova (PD) (IT)

(72) Inventor: Franco Grego, Padova (PD) (IT)

(21) Appl. No.: 17/057,159

(22) PCT Filed: May 20, 2019
(86) PCT No.: PCT/IB2019/054149

§ 371 Date: Nov. 20, 2020

(30) Foreign Application Priority Data
May 23, 2018 (IT) 102018000005638

(51) Int. Cl. A61F 2/07

(52) U.S. Cl. CPC A61F 2/07; A61F 2002/061

(57) ABSTRACT

A fenestrated endoprosthesis for the correction of aortic arch aneurysms comprising: a stent (20) having a first oblong lateral hole (23) having an area equal to the total area occupied by the innominate artery (11) and by the carotid artery (12); said stent (20) comprises a metal skeleton (21) covered with a fabric (22); characterized by comprising a flexible sac (30) that covers said stent (20) covered with fabric (22); said sac (30) comprises a second lateral hole (32) having an area larger than said first hole (23), so that the edges (26) of said stent (20) covered with fabric (22) that delimit said first hole (23) are visible; the edges (33) of said sac (30) that delimit the hole (32) are welded to said stent (20) covered with fabric (22); a volume (31) is created between said stent (20) covered with fabric (22) and said sac (30); said volume (31) is filled with a polymer.

The present invention relates to a fenestrated endoprosthesis for the correction of aneurysms of the aortic arch.

Conventional treatment with open surgery and treatment used with fenestrated or branched endoprostheses still has a high mortality and morbidity rate. In open surgery it is essential that the general clinical conditions of the patient allow a surgical intervention of this magnitude (hypothermia, cardiocirculatory arrest, extracorporeal circulation, cerebral perfusion techniques, prosthetic reconstruction of the aortic arch with re-implantation of the supra-aortic trunks, etc.) with acceptable mortality and morbidity risk.

Endovascular surgery, which originated with the precise intention of reducing invasiveness in the treatment of arterial diseases, has extended the range of action allowing the treatment also of patients suffering from the disease of aneurysms of the aortic arch who would otherwise be untreatable with conventional techniques. However, a reduction in invasiveness does not always go hand in hand with lower perioperative and postoperative morbidity. For example, the stroke represents the most serious complication and if an attempt has been made in open surgery to reduce its incidence with perioperative cerebral reperfusion, it is difficult to prevent in endovascular surgery, as the implantation of fenestrated or branched endoprosthesis on the aortic arch requires direct retrograde access at the level of more than one supra-aortic arterial trunks and the passage therein of guidewires or catheters, potentially dangerous as capable of causing parietal damage (dissection) or of dislodging fragments of parietal atherosclerotic lesions, sources of possible embolic phenomena with terminal destination in the intracranial circulation.

There are many ongoing studies by all manufacturers of endoprostheses for the correction of both thoracic (ascending thoracic aorta, aortic arch, descending thoracic aorta) and abdominal aortic aneurysms, but with regard to the aortic segment relating to the arch, considerable difficulties are encountered relating to the use of multi-modular branched endoprostheses, which are of considerable technical complexity in the implantation step and are not without pen-operative complications, due to the need to navigate with introducers, guidewires and covered stents inside the supra-aortic trunks.

The object of the present invention is to provide a fenestrated endoprosthesis for the correction of aortic aneurysms that is simple.

Another object is to provide a reliable fenestrated endoprosthesis for the correction of aortic aneurysms.

In accordance with the present invention, these objects and others yet are achieved by a fenestrated endoprosthesis for the correction of aneurysms of the aortic arch, comprising: a stent having a first oblong lateral hole having an area equal to the total area occupied by the innominate artery and by the carotid artery; said stent comprises a metal skeleton covered with a fabric; characterized by comprising a flexible sac that covers said stent covered with fabric; said sac comprises a second lateral hole having an area larger than said first hole, so that the edges of said stent covered with fabric that delimit said first hole are visible; the edges of said sac that delimit the hole are welded to said stent covered with fabric; a volume is created between said stent covered with fabric and said sac; said volume is filled with a polymer.

Further features of the invention are described in the dependent claims.

The advantages of this solution with respect to prior art solutions are various.

For implantation of the endoprosthesis in accordance with the present invention no passage at the level of the single supra-aortic trunks is required, thereby reducing the embolic risk source mentioned above.

Aortic arch aneurysms can be of atherosclerotic nature, which by aetiology and natural history do not differ from aneurysms involving the whole aorta, thoracic and abdominal, less frequently are caused by congenital degenerative disease of the arterial wall (Collagenopathies, Marfan Syndrome, Cystic medial necrosis, etc.) or can be the natural evolution of a parietal dissection process, starting from the ascending thoracic aorta, the development of which is always towards aneurysm dilation of the aortic arch and of the descending thoracic aorta, naturally if not treated in the acute phase.

In accordance with the present invention, two principles are exploited: the fenestration principle and the endobag principle (filling of the aneurysm sac), used at abdominal level to prevent the onset of type II endoleaks originating from the lumbar aortic, inferior mesenteric and sacral aneurysm.

The endoprosthesis of the invention consists of a fenestrated endoprosthesis, with fenestration produced at the site of origin (of the supra-aortic trunks) of the innominate and left common carotid arteries. The entire surface of the endoprosthesis is surrounded and covered by a sac (endobag), which once filled with polymers occupies all the free areas between the endoprosthesis and the aortic wall, with the exclusion of the fenestrated area, and preventing perfusion of the aneurysm sac.

The features and advantages of the present invention will be apparent from the following detailed description of a practical embodiment thereof, illustrated by way of non-limiting example in the accompanying drawings, wherein:

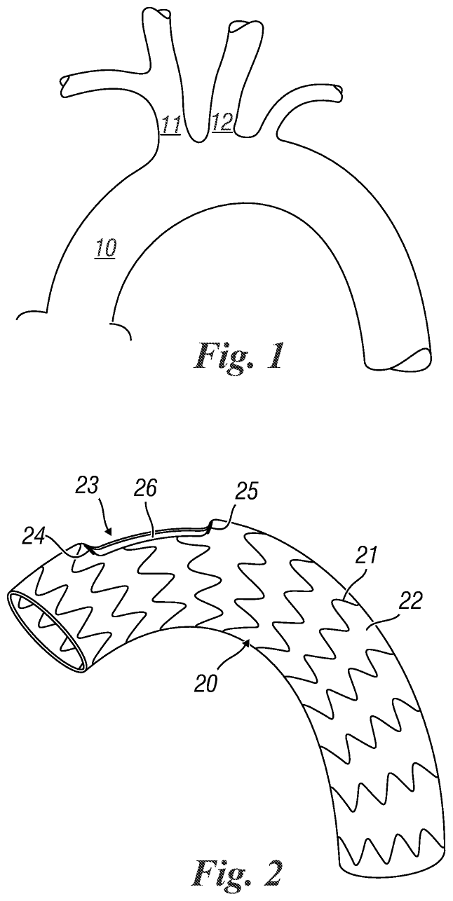

FIG. 1 schematically shows a normal supra-aortic trunk;

FIG. 2 schematically shows a covered stent in accordance with the present invention;

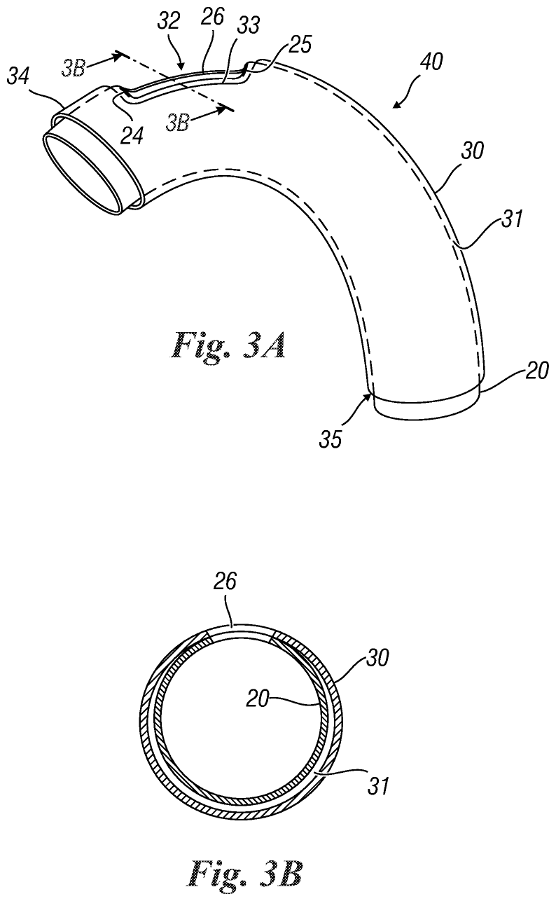

FIGS. 3a and 3b schematically show a covered stent inclusive of an external sac, in a lateral and sectional view, in accordance with the present invention;

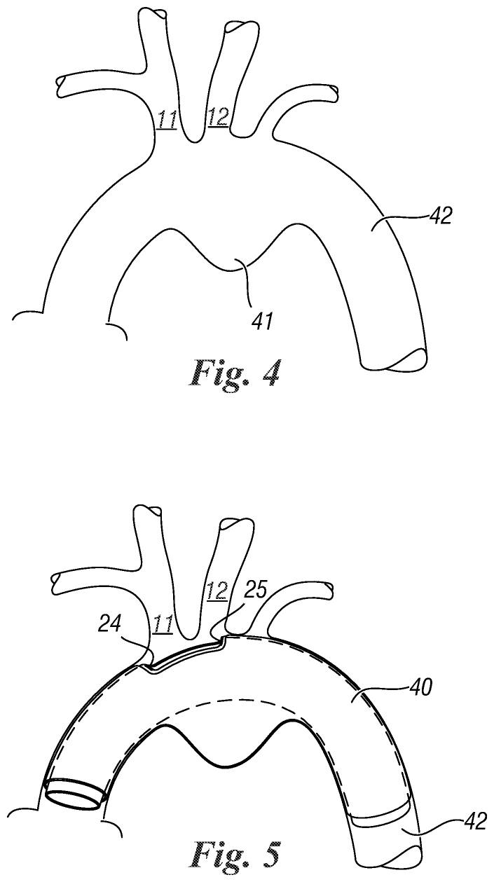

FIG. 4 schematically shows a supra-aortic trunk with an aneurysm;

FIG. 5 schematically shows a covered stent inclusive of an external sac placed in situ in accordance with the present invention.

The endoprosthesis comprises a covered stent 20 that has a metal skeleton 21, for example made of nitinol covered in a fabric 22, for example expanded polytetrafluoroethylene (PTFE).

The covered stent 20 has, in addition to the terminal holes, an oblong lateral hole 23 having an area equal to the total area of the aortic segment 10 occupied by the innominate artery 11 and by the left common carotid artery 12.

Two radiopaque orientation markers 24 and 25 placed at the end edges of said first oblong hole 23 must be defined on the covered stent 20, so that at the time of its positioning they are at the external origins of the innominate artery 11 and of the left common carotid artery 12.

The covered stent 20 is further covered externally by a sac 30, to form an endoprosthesis 40 in accordance with the present invention.

The sac 30 is substantially a flexible tube made of polyurethane, with inner sleeve made of polyester included.

An internal tubular volume 31 is created between the covered stent 20 and the sac 30, which are placed coaxially.

The sac 30 also has a lateral hole 32 coaxial to the hole 23 of the covered stent 20, having an area slightly larger than the total area occupied by the innominate artery 11 and by the carotid artery 12, so that they project from under the edges 26 of the covered stent 20 that delimit the hole 23.

The edges 33 of the sac 30 that delimit the hole 32 are welded to the covered stent 20 with a polyethylene suture.

The edges 34 of one end of the sac 30 are also welded to the covered stent 20, so as to have only one inlet 35 in the volume 31 that is created between the covered stent 20 and the sac 30.

If an aneurysm 41 is present, the endoprosthesis 40 is inserted into the aorta 41 by means of conventional methods, such as a release catheter.

The endoprosthesis 40 is positioned, checking its correct positioning, so that the two markers 24 and 25 are at the external origins of the innominate artery 11 and of the left common carotid artery 12.

Now, in accordance with the present invention, the volume 31 included between the covered stent 20 and the sac 30 must be filled with an amorphous material, such as for example a polymer that maintains a plastic state, such as the polymer used in breast implants, for example composed of a biocompatible and biostable blend based on polyethylene glycol (PEG).

Before inserting the polymer, it is preferable to measure the volume of polymer that is to be inserted, which is calculated on the basis of the difference between the overall volume of the aneurysm arch and the volume of the endoprosthesis 40 (i.e., the free space between endoprosthesis and aortic walls) with a transducer in the circuit that also allows the filling pressure to be measured as further verification.

In order to take the measurement a saline solution can be inserted and the volume inserted is calculated. In fact, the sac 30 is completely closed also around the fenestration, with the exception of a small inlet 35 (the edges can be sutured partially to leave this inlet).

Subsequently, a volume of polymer equal to the volume of saline solution, previously measured, will be inserted.

The polymer is inserted and the inlet 35 is then sutured completely by means of polyethylene.

The endoprosthesis thus conceived is susceptible to numerous modifications and variants, all falling within the scope of the inventive concept; moreover, all details can be replaced by technically equivalent elements.

1. A fenestrated endoprosthesis for the correction of aortic arch aneurysms comprising: a stent (20) having a first oblong lateral hole (23) having an area equal to the total area occupied by the innominate artery (11) and by the carotid artery (12); said stent (20) comprises a metal skeleton (21) covered with a fabric (22); characterized by comprising a flexible sac (30) that covers said stent (20) covered with fabric (22); said sac (30) comprises a second lateral hole (32) having an area larger than said first hole (23), so that the edges (26) of said stent (20), covered with fabric (22) that delimit said first hole (23), are visible; the edges (33) of said sac (30) that delimit the hole (32) are welded to said stent (20) covered with fabric (22); between said stent (20) covered with fabric (22) and said sac (30) a volume (31) is created; said volume (31) is filled with a polymer.

2. The endoprosthesis in accordance with claim 1, characterized in that said stent (20) covered with fabric (22) comprises two radiopaque orientation markers (24) and (25) positioned at the end edges of said first oblong hole (23).

3. The endoprosthesis in accordance with claim 1, characterized in that said polymer is a thermosetting polymer composed of a biocompatible and biostable blend based on polyethylene glycol (PEG).

4. The endoprosthesis in accordance with claim 1, characterized in that said sac (30) is made of polyurethane.

5. The endoprosthesis in accordance with claim 1, characterized in that the edges of said sac (30) are welded at proximal level (35) and at distal level (34) to said stent (20) covered with fabric (22).

6. The endoprosthesis in accordance with claim 5, characterized in that the edges (33) of said sac (30) are welded to said stent (20) covered with fabric (22) by means of a polyethylene suture.

7. The endoprosthesis in accordance with claim 1, characterized in that said fabric (22) is made of expanded polytetrafluoroethylene (PTFE).

8. A method for producing a fenestrated endoprosthesis for the correction of aortic arch aneurysms comprising the steps of: providing a stent (20) comprising a metal skeleton (21) made of nitinol covered in a fabric (22); making a first oblong lateral hole (23) in said stent (20) covered with fabric (22) having an area equal to the total area occupied by the innominate artery (11) and by the carotid artery (12); covering said stent (20) covered with fabric (22) with a flexible sac (30); making a second lateral hole (32) in said sac (30) having an area larger than said first hole (23), so that the edges (26) of said stent (20), covered with fabric (22) that delimit said first hole (23), are visible; welding the edges (33) of said sac (30) that delimit the hole (32) to said stent (20) covered with fabric (22); welding the edges of said sac (30) at distal level (34) of said stent (20) covered with fabric (22); filling a volume (31) that is created between said stent (20) covered with fabric (22) and said sac (30) with a polymer; welding the edges of said sac (30) at proximal level (35) of said stent (20) covered with fabric (22).
